SEQUENCE LISTING (9 sequences)

SEQ ID NO: 1 - DNA, 12 bp, Artificial Sequence
  Description of Artificial Sequence Synthetic oligonucleotide
atggatggatgg

SEQ ID NO: 2 - DNA, 16 bp, Artificial Sequence
  Description of Artificial Sequence Synthetic oligonucleotide
aaaaaaaaaaaaaaaa

SEQ ID NO: 3 - DNA, 14 bp, Artificial Sequence
  Description of Artificial Sequence Synthetic oligonucleotide
cgcgcgcgcgcgcg

SEQ ID NO: 4 - DNA, 10 bp, Artificial Sequence
  Description of Artificial Sequence Synthetic oligonucleotide
atgtcatggt

SEQ ID NO: 5 - DNA, 10 bp, Artificial Sequence
  Description of Artificial Sequence Synthetic oligonucleotide
tgatgaccat

SEQ ID NO: 6 - DNA, 15 bp, Artificial Sequence
  Description of Artificial Sequence Synthetic oligonucleotide
tgatgaccatgacat

SEQ ID NO: 7 - DNA, 22 bp, Artificial Sequence
  Description of Artificial Sequence Synthetic oligonucleotide
ggagtccgacctgcttcattat

SEQ ID NO: 8 - DNA, 26 bp, Artificial Sequence
  Description of Artificial Sequence Synthetic oligonucleotide
ggagtccgacctgcttcattatcaca

SEQ ID NO: 9 - DNA, 15 bp, Artificial Sequence
  Description of Artificial Sequence Synthetic oligonucleotide
gtacnnnnnnnttcg

(19) United States
(12) Patent Application Publication
(10) Pub. No.: US 2016/0376584 A1
(43) Pub. Date: Dec. 29, 2016

(54) METHOD AND APPARATUS FOR DUAL SOLID PHASE NUCLEIC ACID SYNTHESIS

(71) Applicant: Genesis DNA Inc., Wilmington, DE (US)

(72) Inventors: David Glass, Stanford, CA (US); Jefferson Clayton, Somerville, MA (US)

(21) Appl. No.: 15/193,896

(22) Filed: Jun. 27, 2016

Related U.S. Application Data
(60) Provisional application No. 62/186,260, filed on Jun. 29, 2015.

(51) Int. Cl. C12N 15/10; B01L 3/00

(52) U.S. Cl. CPC C12N 15/1093; B01L 3/502761; B01L 2300/0663; B01L 2200/027; B01L 2200/143; B01L 2300/0867; B01L 2400/043

(57) ABSTRACT

Provided herein are methods and apparatuses for synthesizing nucleic acids having a predefined sequence through enzymatic elongation. In some embodiments, the methods and/or apparatuses comprise controlled manipulation of solid objects with respect to a solid substrate comprising an oligonucleotide template array.

### CROSS REFERENCE TO RELATED APPLICATION

This application claims the benefit of and priority to U.S. Provisional Application No. 62/186,260, filed Jun. 29, 2015, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jul. 27, 2016, is named 2011943-0004_SL.txt and is 2,486 bytes in size.

### FIELD OF THE INVENTION

The invention relates broadly to the field of synthetic biology and more specifically to the field of nucleic acid synthesis. The present invention relates to methods and apparatuses for synthesizing nucleic acids having a predefined sequence through enzymatic elongation and the controlled manipulation of solid objects with respect to a solid substrate comprising an oligonucleotide template array.

### BACKGROUND

Virtually every experiment in biology begins with the right DNA: a small primer, a gene, a plasmid, a virus, or even a genome. Weeks or even months can be spent in laboratories obtaining the right DNA before even the first real experiment is carried out. It is no surprise then that the commercial synthesis beginning a few decades ago of even small oligonucleotides (“oligos”), or fragments of single-stranded DNA (ssDNA) up to 20 or, more recently, 200 nucleotides (nt) revolutionized molecular biology, allowing scientists to obtain or assay genes of interest by polymerase chain reaction (PCR).

More recently, gene synthesis, which delivers double-stranded DNA (dsDNA), is revolutionizing the way that biologists solve today's questions. Genes can be purchased even if the organism the gene comes from is not readily available, as long as the sequence is in online databases. Unnatural sequences are becoming increasingly important as well, for the growing field of synthetic biology, which aims to design biological systems to perform novel tasks, such as logic processing, environmental remediation, biotherapeutic performance, and programmed tissue engineering.

Despite recent advances in gene synthesis techniques, the use of synthetic genes or even genomes is limited due to high prices, long turnaround times, and variable synthesis success rates depending on the sequence. New methods are desired to circumvent sequence-dependent complexities and lower gene synthesis costs.

### BRIEF SUMMARY OF THE INVENTION

Provided herein include a method and associated device to provide fast gene synthesis at low cost with low error rates. The present invention relates broadly to the field of molecular biology, and more specifically to field of synthetic biology, and even more specifically to the field of nucleic acid synthesis. The present invention relates to methods and apparatuses for synthesizing nucleic acids having a predefined sequence, optionally comprising specified variations at given points in the sequence, through enzymatic elongation and the controlled manipulation of solid objects with respect to a solid substrate comprising oligonucleotide template molecules. In some embodiments, a predefined sequence is a partially predefined sequence. For example, in some embodiments, a predefined sequence comprises a first segment having a predefined sequence, and a second segment whose sequence is not predefined. In some embodiments, a second segment has a not predefined sequence which is randomized or partially randomized.

According to an embodiment of the invention, a method of synthesizing in cycles a collection of nucleic acid sequences attached to a solid object is provided. In some embodiments, the cycles comprise the total elongation of a collection of nucleic acid sequences attached to a solid object, carried out per cycle in steps of (1) moving the solid object to a position with respect to an oligonucleotide array, (2) hybridization of the object-attached nucleic acid sequence to an array-bound nucleic acid sequence at the specified location, and (3) enzymatic elongation of the object-attached nucleic acid sequences by, for example, DNA polymerase in a manner following base pair complementarity with respect to the array-bound template sequence. In some embodiments, the order of such cycles is determined by computer software and based on the desired fully elongated nucleic acid sequence.

According to an embodiment of the invention, a method of synthesizing nucleic acids that comprises the use of an array of oligonucleotides on a glass or silicon or other substrate (often termed a “microarray”) is provided. Methods and apparatuses provided herein may involve an array with a large number of oligonucleotide sequence features. For example, an array may comprise features in quantities of 100, or 1,000, or 10,000, or 100,000 or 1,000,000 or more. In some embodiments, some features comprise a plurality of identical oligonucleotide sequences, and some features comprise a plurality of oligonucleotide sequences with specified variability in part or all of the oligonucleotide sequence. In some embodiments, template oligonucleotides are bound to solid substrates via biotin/streptavidin, maleic anhydride/amine, thiol/maleimide, or other covalent or noncovalent bond with or without a hydrocarbon, polyethylene glycol, or other spacer on either the oligonucleotides or solid substrates.

According to an embodiment of the invention, a method of synthesizing nucleic acids that comprises a solid object or objects which can be moved precisely within a microfluidic chamber with respect to a solid substrate is provided. In some embodiments, such objects may be, but are not limited to, small superparamagnetic beads or dielectric beads. For example, the size of the objects may be less than 10 microns, less than 5 microns, or about 1 micron, or smaller. In some embodiments, a solid object comprises a paramagnetic, superparamagnetic, ferromagnetic, dielectric, or other bead or disc about 100 microns, 50 microns, 10 microns, 5 microns, 1 micron or smaller in diameter.

According to an embodiment of the invention, a method of manipulating one or more solid objects with attached nucleic acids simultaneously and independently relative to an oligonucleotide template substrate in two or three dimensions is provided. Such a method comprises manipulating object position using a computer or microcomputer or equivalent integrated circuit that controls object detection and positioning systems. In some embodiments, the positioning system may comprise optoelectronic tweezers, optical tweezers with or without a micromirror array, dielectrophoresis, acoustic trapping, microfluidics, on-chip magnetics, off-chip magnetics, electromagnetics, dielectrophoretic trapping with or without optical or microfluidic control, and mechanical motion of the substrate. In some embodiments, the detection system may comprise a CCD detector or camera with or without additional optical components or lenses. In some embodiments, the detection system may comprise a CMOS or CCD detector or other camera with or without additional optical components or lenses.

According to an embodiment of the invention, the number of objects manipulated may be increased to 10, 100, 1,000, 10,000, or 100,000 or more by using massively parallel manipulation technologies such as, but not limited to, optoelectronic tweezers, optical tweezers with a micromirror array, dielectrophoresis, acoustic trapping, microfluidics, on-chip magnetics, off-chip magnetics, electromagnetics, dielectrophoretic trapping with or without optical or microfluidic control, and mechanical motion of the substrate.

According to an embodiment of the invention, a method of providing reaction fluids, rinsing fluids, and solid objects with attached nucleic acids to a microfluidic chamber adjacent to an oligonucleotide array is provided. In some embodiments, reaction fluid may comprise enzymes (e.g. polymerase, ligase, etc.), nucleic acid primers, deoxynucleotide triphosphates (dNTPs), buffer, etc. or any combination thereof. In some embodiments, a microfluidic chamber may comprise a microfluidic chip positioned proximate to a substrate with channels for loading and unloading reaction fluids, rinsing fluids and solid objects with attached nucleic acids. In further embodiments, microfluidic channels may offload solid objects with attached nucleic acids from a microfluidic chamber to a separate chamber in preparation for post-synthesis processing.

According to an embodiment of the invention, post-synthesis processing may comprise the steps of amplification and filtering of the synthesized nucleic acids attached to solid objects, for example, to remove sequences that contain errors. In some embodiments, synthesized nucleic acids may be amplified while attached to solid objects or after being cleaved from solid objects and remaining in solution. In some embodiments, nucleic acids may be filtered post-synthesis by length. In some embodiments, synthesized nucleic acids may be hierarchically assembled into longer sequences. In some embodiments, parts of the synthesized sequence may be cleaved to facilitate post-synthesis uses such as, for example, hierarchical assembly or cloning. In some embodiments, synthesized nucleic acids may be cloned into DNA plasmid vectors and in some embodiments may be transferred to a host organism.

### BRIEF DESCRIPTION OF THE DRAWINGS

FIG. 1 illustrates a schematic of an embodiment of a device used to implement the synthesis methods provided herein. Solid objects, such as but not limited to dielectric beads, are loaded into a microfluidics chamber (103) from a reservoir (104) along with reagents. One face of the microfluidics chamber (103) may comprise a detection system (101) and a positioning system (102), which may be separate from or combined into a single system with (101). The detection system may comprise but is not limited to a lensless optical setup that comprises a CMOS imaging chip for providing feedback to a computer controlling the positioning system, while the positioning system supplies the physical forces, electromagnetic or otherwise, by which beads are manipulated. The opposite face of the microfluidics chamber comprises a oligonucleotide template array (110) comprising possible oligonucleotide types (see FIG. 3). Each location (“feature”) on the array, for example (106), (107) and (108), comprises a plurality of oligos, (109), that is unique to that array location. Once synthesis is complete, the beads are shuttled through exit channel (105) to a separate area for post-processing.

FIG. 2A illustrates an example low-throughput embodiment of a device used to implement the synthesis methods provided herein. A superparamagnetic bead (203) is manipulated to locations on an oligonucleotide template array (202) composed of possible oligonucleotide types (see FIG. 3). Each location on the array, for example (204), (205) and (206), comprises a plurality of oligos unique to that array location. A detection system (201) sits adjacent to an oligonucleotide array and is used for closed-loop feedback control. The detection system, for example, comprises a lensless optical setup that comprises a CCD detector or CMOS imaging chip. The bead is physically manipulated to its next location on the array (202) via electromagnets (207) controlled by a computer and microcontroller. In a feedback algorithm, the bead's current location is captured by the detection system and transmitted to a computer, which knows where the bead must go next based on the predefined nucleic acid sequence. A microcontroller then receives instructions from a computer about which magnets to turn on or off and at what strength, which are periodically adjusted based on updated input from the detection system until the bead comes to rest at its next destination.

FIG. 2B illustrates an example high-throughput embodiment of a device used to implement the synthesis methods provided herein. A detection system (201) comprising of, for example, a CMOS imaging chip, rests adjacent to positioning system (218) and a surface array (202) composed of possible oligo types (see FIG. 3). Each location on the surface array, for example, (204), (205) and (206), comprises a plurality of oligos unique to that array location. The detection system (201) and the positioning system (218) are separate but may be combined into a single system. The positioning system is comprised of an array of electrodes, connected to a computer, which is used to perform massively parallel and independent manipulation of dielectric beads via dielectrophoretic motion; the number of beads simultaneously and independently manipulated may be 10, 100, 1000, 10000, or more, with (208), (209), (210), (211), (212) shown as example beads comprised within their respective dielectrophoretic cages (214), (215), (217), (216), and (213). A closed-loop feedback control based on input to a computer from the detection system and selective output to electrodes on the positioning system is implemented to achieve simultaneous and independent syntheses of up to as many predefined nucleic acid sequences as there are beads.

FIG. 3 illustrates a non-limiting example of an oligonucleotide template array with possible oligonucleotide sequence types (see claim 8). A surface (307), (for example, glass) is comprised of an array of individual “features” that are clusters of single-stranded DNA, with (301), (302), (303), (304), (305), (306) shown as examples (SEQ ID NOS 2-3 and 9, respectively, in order of appearance). Each feature may comprise an identical cluster of one of the 4\hat{L} possible sequences of length L (308) or a homopolymer or tandem repeat (309) or a library of strands where part of the sequence denoted by “NNN . . . ” is randomized among the library (310) or part of the sequence contains non-canonical or universal nucleotides denoted by X, Y (311) or any combination of these possibilities.

FIG. 4 illustrates an example embodiment of the process of annealing and extension that occurs during a single cycle at a specified feature on the oligonucleotide template array. In step I, the bead (402) is moved to a feature on the surface (401). The growing strand (403) connected to the bead anneals by complementary base pairing to the strand (404) on the surface. In some embodiments, the surface strand (404) ends on the 3′ end with a dideoxynucleotide denoted by an asterisk (405). In some embodiments, the surface strand (404) ends on the 3′ end with a phosphate, inverted dT, or other 3′ chemical modifications to prevent extension by polymerases. In step II, DNA polymerase (406) binds to the partially double-stranded DNA complex (403)-(404) and extends the bead-bound strand (403) in a manner complementary to the surface-attached sequence (404). The dideoxynucleotide (405) prevents extension of the surface bound strand (404). In some embodiments, a phosphate, inverted dT, or other 3′ chemical modifications prevents extension of the surface bound strand (404). In step III, the polymerase detaches, leaving the newly extended strand (407) which constitutes part of the synthesized DNA. Note that the template oligonucleotide (404) is not modified during the process exemplified in this figure. FIG. 4 I, II and III disclose SEQ ID NOS 4-5, 4 and 6, respectively, in order of appearance.

FIG. 5 illustrates a non-limiting example of post-synthesis processing methods that may be used with the herein described synthesis device and method. A single bead (501) comprising a variety of synthesized DNA (containing both correct and erroneous sequences) is subject to polymerase chain reaction (502), yielding a large number of double stranded DNA molecules (503). These are filtered by length, for example, via HPLC or capillary electrophoresis (504), leaving a population of DNA molecules of only the desired length (505). This population is then assembled (508) by, for example, Gibson assembly, together with a linearized plasmid vector (507) and sequences from length-filtered DNA populations from other beads (506), used for hierarchical assembly of longer sequences. This yields circularized plasmid DNA (509), which may be transformed into bacteria for further amplification, followed by sequencing and isolation (510) of a sequence-perfect library of DNA plasmids (511). In other embodiments, post-processing may include polony-picking with or without non-destructive sequencing protocols.

FIG. 6 illustrates microscopic time lapse of moving bead (dark object surrounded by the big circles) towards successive target locations (indicated by the small circles).

FIG. 7 illustrates Sanger sequence confirmation for extension of a primer oligo attached to a bead after templated addition of the four nucleotides CACA. Figure discloses SEQ ID NOS 7-8, respectively, in order of appearance.

FIG. 8 illustrates successive addition of 4 nt in 4 consecutive extension cycles.

FIG. 9 illustrates an example device for controlled manipulation of superparamagnetic beads relative to an oligonucleotide template surface.

REFERENCE NUMERALS IN THE DRAWINGS

| | | | |
|---|---|---|---|
| 101 | detection system | 102 | positioning system |
| 103 | fluidics system/microfluidics | 104 | solid object reservoir |
| | chamber | | |
| 105 | exit channel to post- | 106, 107, 108 | unique oligonucleotide |
| | processing | | template array feature |
| | | | location |
| 109 | plurality of oligonucleotides | 110 | oligonucleotide template |
| | | | array |
| 201 | detection system | 202 | oligonucleotide template |
| | | | array |
| 203 | solid object, e.g. bead, with | 204, 205, 206 | unique array feature |
| | attached nucleic acids | | location |
| 207 | electromagnet | 208, 209, 210, | independently controlled |
| | | 211, 212 | solid objects |
| 213, 214, 215, | independent dielectrophoretic | 218 | positioning system |
| 216, 217 | cages surrounding solid | | |
| | objects | | |
| 301, 302, 303, | unique array feature location | 307 | oligonucleotide array |
| 304, 305, 306 | comprising plurality of | | |
| | oligonucleotide | | |
| 308 | 4possible sequences of | 309 | homopolymer or tandem |
| | length L | | repeat sequences |
| 310 | library of strands with | 311 | any sequence combination |
| | randomized subsequence | | of 308, 309, 310 |
| 401 | oligonucleotide template | 402 | solid object, e.g. bead, with |
| | array surface | | attached nucleic acids |
| 403 | growing strand | 404 | oligonucleotide template |
| | | | array feature on surface |
| 405 | dideoxynucleotide cap or | 406 | DNA polymerase |
| | similar modification | | |
| 407 | newly extended strand | 501 | single solid object, e.g. |
| | | | bead, with attached nucleic |
| | | | acids |
| 502 | polymerase chain reaction | 503 | post-amplification plurality |
| | | | of dsDNA molecules |
| 504 | filtering protocol, e.g. HPLC | 505 | plurality of DNA molecules |
| | or capillary electrophoresis | | containing only proper |
| | | | length sequences |
| 506 | length-filtered DNA | 507 | linearized plasmid vector |
| | populations from other solid | | |
| | objects with attached nucleic | | |
| | acids | | |
| 508 | assembly protocol, e.g. | 509 | circularized plasmid DNA |
| | Gibson assembly | | |
| 510 | sequencing and isolation | 511 | sequence-perfect library of |
| | protocol | | DNA plasmids |

### DETAILED DESCRIPTION OF THE INVENTION

Provided herein are apparatuses and related methods for the synthesis of nucleic acids. Current state of the art methods for synthesis of nucleic acids have difficulties producing certain types of nucleic acid sequences depending on the precise ordering of the nucleotide monomers in the nucleic acid polymer sequence. The methods and associated devices described herein are capable of synthesizing difficult sequences such as, but not limited to, homopolymeric repeats, tandem repeats, high GC content, low GC content (collectively, “complex sequences”), and nucleic acid libraries in which part of a sequence is purposefully randomized or partially randomized to generate variation within a plurality of otherwise identical synthesized nucleic acid molecules. A general method used for synthesizing most simple sequences not comprising such complex sequences or libraries is described herein first, followed by embodiments of methods and associated devices capable of synthesizing complex sequences and nucleic acid libraries.

Additionally, current state of the art methods consume single-stranded DNA oligonucleotides in the synthesis of nucleic acid double-stranded DNA molecules, which places a lower bound on the cost of nucleic acid synthesis. The methods and devices described herein uses oligonucleotides only catalytically, providing a substantial decrease in nucleic acid synthesis cost relative to the state of the art.

The following descriptions illustrate some examples of certain embodiments of the invention, and are not meant to be limiting. Common components among example embodiments comprise a detection system, a positioning system, a microfluidics network, a solid substrate comprising an oligonucleotide template array, and a synthesis chamber. Each component is independently functional and may interact with or be combined with other components; the collective action of the components comprises the methods implemented by an embodiment of the invention for the synthesis of nucleic acids. Not all systems may be physically distinct.

One aspect of the technology provided herein relates to the design of a positioning system for the controlled manipulation of solid objects with respect to a solid substrate comprising an oligonucleotide template array. In some embodiments, the positioning system involves moving solid objects to a desired feature location. In some embodiments, the positioning system involves moving a solid substrate so that solid objects are positioned at the desired feature location. In some embodiments, the positioning system involves moving both solid objects and solid substrates so that solid objects are positioned at the desired feature location. The collective motion of the solid objects from feature to feature on the template array supplies the total synthesis of the attached nucleic acids from primer sequences to a predefined sequence. In some embodiments, the solid objects manipulated with respect to the template array surface are, for example but not limited to, superparamagnetic beads or dielectric beads. The size of the objects may be less than 10 microns, less than 5 microns, or about 1 micron, or smaller.

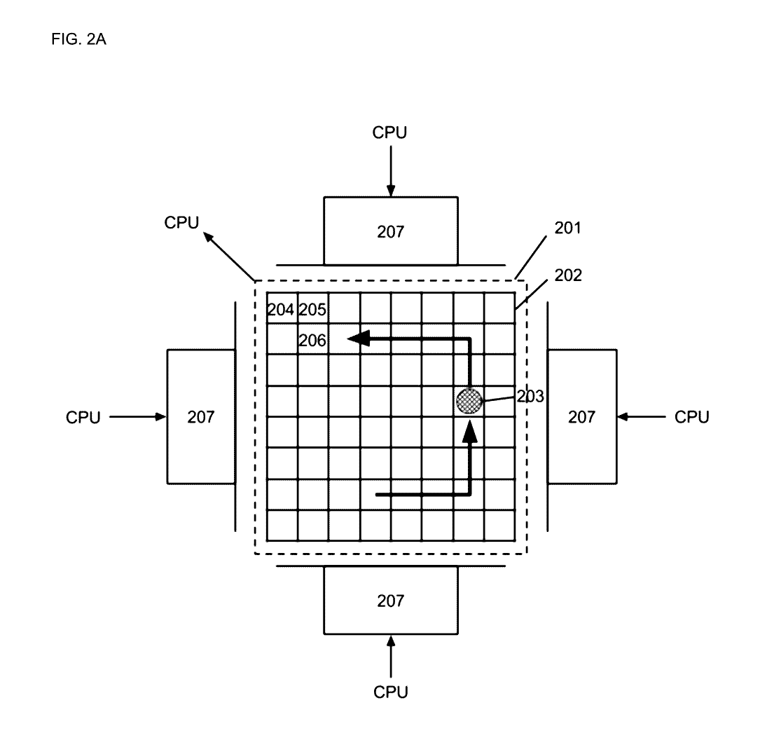

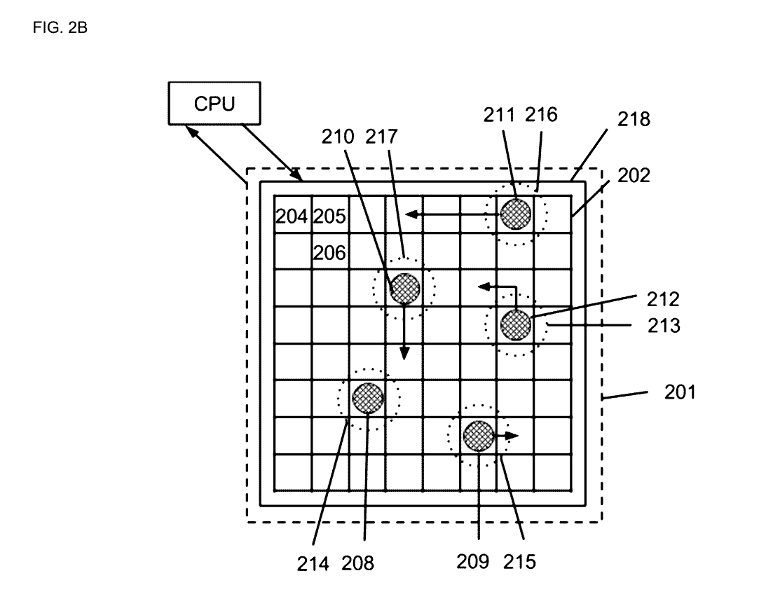

In an example low throughput embodiment, multiple electromagnets may be used to attract one or a few beads in up to 3 dimensions (FIG. 2A); in an example high throughput embodiment, an array of electrodes is used to create dielectrophoretic cages around 10, 100, 1,000, or 10,000 or more solid objects which may then be independently and simultaneously moved along electric potential gradients in up to 3 dimensions (FIG. 2B). In some embodiments, the positioning system may comprise optoelectronic tweezers, optical tweezers with or without a micromirror array, dielectrophoresis, acoustic trapping, microfluidics, on-chip magnetics, off-chip magnetics, electromagnetics, dielectrophoretic trapping with or without optical or microfluidic control, and mechanical motion of the substrate.

In some embodiments, the positioning system is connected to a computer or equivalent integrated circuit. The computer runs a closed-loop feedback control algorithm which accepts input from a detection system about the solid objects' current locations and which produces an output that operates magnets or electrodes in such a way that they physically manipulate the beads to the next array location. In some embodiments, the detection system may comprise, for example, a lensless optical system including a CMOS imaging chip or CCD detector, but may alternatively utilize lenses or other means of detection.

In some embodiments, a fluidics network, which describes a chamber in which synthesis of a target sequence takes place, provides the means of loading solid objects with attached nucleic acids in from a reservoir and off-loading them to a separate area for post-processing. Beads are physically manipulated within the fluidics network, which can be a microfluidics network. In some embodiments, the liquid medium held in the fluidics network comprises all the necessary materials needed for synthesis, such as but not limited to polymerases, dNTPs, buffer, single-strand binding proteins, and ligases. The inside faces of the fluidics chamber comprise the oligonucleotide template array, the physical components of the positioning system and detection system, or all of the above if not located on the outside faces of the chamber.

In some embodiments, nucleic acid synthesis occurs adjacent to an oligonucleotide template array and within a fluidics chamber filled with reagents such as but not limited to enzymes, buffers, and deoxynucleotide triphosphates (dNTPs). Nascent nucleic acid polymers may be attached to solid objects, for example dielectrophoretic or superparamagnetic beads, which are present in a fluidics chamber. Nascent nucleic acid polymers may be attached to solid objects, for example paramagnetic, superparamagnetic, ferromagnetic, dielectric, or other bead or disc, which are present in a fluidics chamber. Solid objects with nascent nucleic acid polymers are moved to various positions on an oligonucleotide array by a positioning system (in conjunction with a detection system and computer for closed-feedback control). A synthesis cycle using the device systems is described herein.

In some embodiments, a nucleic acid (e.g., a nascent nucleic acid polymer) is attached to solid objects via biotin/streptavidin, maleic anhydride/amine, thiol/maleimide, or other covalent or noncovalent bond with or without a hydrocarbon, polyethylene glycol, or other spacer on either the nucleic acid polymer or solid objects.

In some embodiments, a universal primer oligonucleotide is loaded onto solid objects prior to synthesis of a desired target sequence. In some embodiments, the primer is not part of a desired sequence, but will be bound to it to simplify post-synthesis processing of synthesized nucleic acids. Primer ends may be used post-synthesis for incorporation into a DNA plasmid, for example, or removed enzymatically. In some embodiments, primers are used which ensure desired sequence compatibility with polymerase chain reaction amplification of synthesized nucleic acids. A separation protocol may be used to ensure that all primers are correctly attached. Non-limiting example attachment protocols that may be used are: non-covalent bonding between biotinylated primers and streptavidin-coated solid objects, bonding between amine-modified primers and epoxy silane or isothiocyanate-coated solid objects, bonding between succinylated primers and aminophenyl or aminopropyl-derivatized solid objects, or bonding between disulfide-modified primers and mercaptosilanized solid objects.

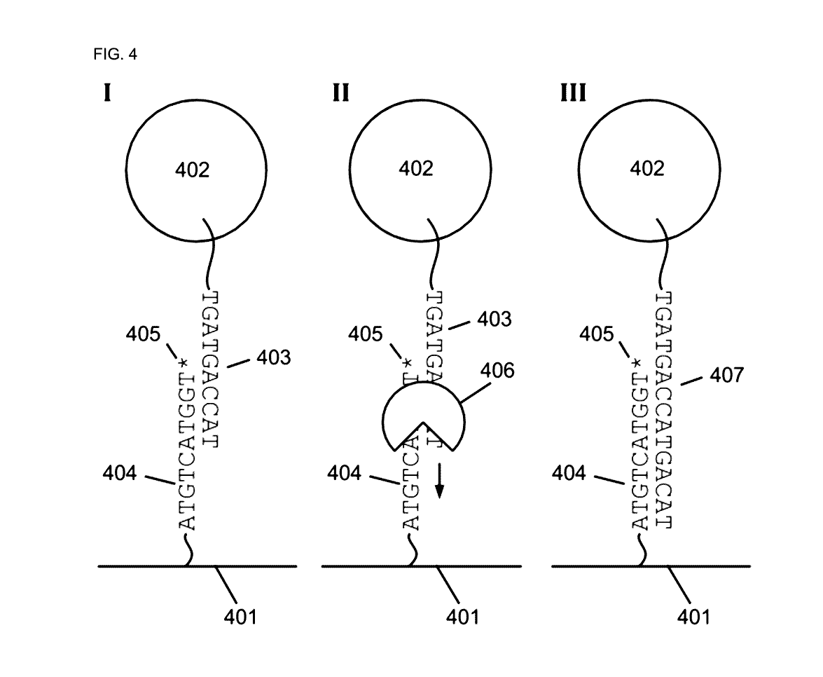

In some embodiments, primary synthesis of a desired target sequence occurs in cycles comprising the following steps. A solid object with attached nucleic acids is moved by a positioning system (in conjunction with a detection system and computer for closed-feedback control) to a particular feature on an oligonucleotide template array. The feature to which the solid object is moved comprises a sequence that is base-pair complementary to the end of the sequence attached to the solid object. Upon hybridization of the solid object-attached sequence and the array-attached sequence, enzymatic elongation by one or more enzymes present in the liquid medium, for example, DNA polymerase, occurs, adding nucleotides complementary to the remaining non-hybridized length of the array-attached sequence (see FIG. 4). Once enzymatic elongation is complete, the cycle is complete and the positioning system manipulates the solid object to the next appropriate feature on the array. In some embodiments, a positioning system may additionally be used to lock the solid object in place with enough precision to suppress Brownian fluctuations during hybridization and enzymatic elongation.

The terms “base pair” or “base pairing,” as used herein, refer to non-covalent binding of two single-stranded nucleic acid molecules or sub-sections of molecules driven by pairwise specificity between two bases. In some embodiments, a base pair is formed between two canonical nucleotides, such as adenine, thymine, guanine, cytosine. In some embodiments, a base pair involves one or two non-canonical nucleotides. Non-canonical nucleotides are natural or synthetic nucleotides other than the four canonical nucleotides. In some embodiments, a non-canonical nucleotide is 2′-deoxyinosine. In some embodiments, a base pair is formed between 2′-deoxyinosine and any of the four canonical nucleotides.

The term “hybridization of the solid object-attached sequence and the array-attached sequence,” as used herein, refer to formation of a duplex region between the solid object-attached sequence and the array-attached sequence. A hybridization can be between a subsection of the solid object-attached sequence and a subsection of the array-attached sequence. The number of base pairs in the duplex region may vary. In some embodiments, the duplex region comprises at least the minimum number of base pairs that are required by the enzyme used in the elongation. In some embodiments, the duplex region comprises 1 base pair. In some embodiments, the duplex region comprises 2 base pairs or more. In some embodiments, the duplex region comprises 3, 4, 5, 6, 7, 8, 9, or 10 base pairs. In some embodiments, the duplex region comprises more than 10 base pairs.

In some embodiments, for the entire synthesis, the synthesis chamber is maintained at a single temperature. For example, the chamber may be held at about 12° C. if using T4 DNA polymerase. No temperature changes are needed to melt the hybridized strands, because the departing solid object exerts a controlled force that pulls the growing strand from the array-attached strand quickly and without any damage to the DNA. In some embodiments, universal nucleotides such as, but not limited to, 2′-deoxyinosine may be included at the 3′ end of surface-attached oligonucleotides to increase hybridization melting temperature.

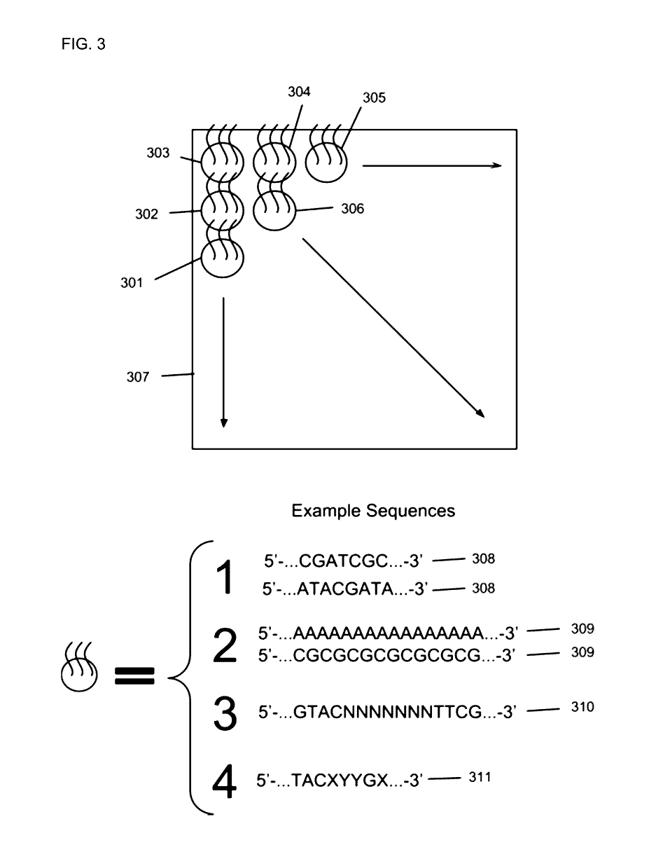

In some embodiments, the space of target sequences that can be synthesized effectively by the method and device herein is determined by the composition of an oligonucleotide array used. The aforementioned simplified method extends a synthesized nucleic acid strand by the same number of nucleotides each cycle, but relaxing this constraint allows improved speed and efficiency for synthesis of complex sequences. Because an oligonucleotide array may include millions of features, a variable extension length is easily accommodated, and when to use variable-length features during synthesis can be easily optimized by the computer. Provided below are certain exemplary embodiments of the optimization (see FIG. 3).

In some embodiments, a set of array features dedicated to synthesizing sequences comprising variable-length homopolymers may be provided. A homopolymer is defined as a stretch of sequence comprising just a single nucleotide, such as AAAA . . . or GGGGG . . . (see FIG. 3). In some embodiments, the array-bound sequences at such features may comprise, for example, binding regions and a variable-length homopolymer region. The binding regions may comprise the reverse complement of a portion of the sequence added to a growing solid object-attached strand in the previous synthesis cycle, or a portion of the free end of the array-bound sequence comprising the array feature to be visited in the next cycle.

In some embodiments, a set of array features dedicated to synthesizing sequences comprising tandem repeats may be provided. Tandem repeats are defined as short sequences that are repeated, such as ATGGATGGATGG . . . (see FIG. 3) (SEQ ID NO: 1). For an array-bound sequence comprising tandem repeats and a solid object-attached sequence comprising the reverse complement tandem repeats, an erroneous binding may occur if the overlap is shifted relative to the intended overlap by an integer multiple of the length of the tandem repeat unit. In some embodiments, erroneous binding issues may be avoided at array features comprising short array-bound sequences, such that there is no way to have shifted binding with the solid object-attached sequences.

In some embodiments, a set of array features dedicated to synthesizing sequences comprising stretches of low GC content may be provided. Low GC content sequences tend to hybridize poorly due to weaker hydrogen bonding compared with non-complex sequences. For synthesizing low GC content sequences, a longer hybridization region may be used to increase the thermodynamic stability of array-attached template DNA and solid object-attached DNA. Additionally, using a single low temperature throughout synthesis may be used as mentioned above, allowing hybridization of low GC content sequences to occur more readily.

In some embodiments, a set of array features dedicated to synthesizing sequences comprising stretches of high GC content may be provided. High GC content sequences dehybridize poorly and form hairpins and other secondary structures in single-stranded DNA due to stronger hydrogen bonding compared with non-complex sequences. Dehybridization may be accommodated through use of mechanical force to pull strands apart, rather than temperature which is used in the state of the art. Secondary structures may be dealt with separately (see for example, [0044]).

In some embodiments, a set of array features may be provided dedicated to synthesizing nucleic acid libraries, which comprise partially randomized sequences within a plurality of otherwise identical molecules. Features on an oligonucleotide array may accommodate libraries by including, for example, oligonucleotide templates comprising either (1) a plurality of molecules in which one or more nucleotides within the plurality are randomized (e.g., ATGNTGC) or (2) non-canonical nucleotides, such as but not limited to 2′-deoxyinosine, which bind to multiple nucleotides with approximately equal affinity (e.g., GCAXCAT). In order to increase variation within a nucleic acid library, multiple synthesis runs with multiple solid objects may be run.

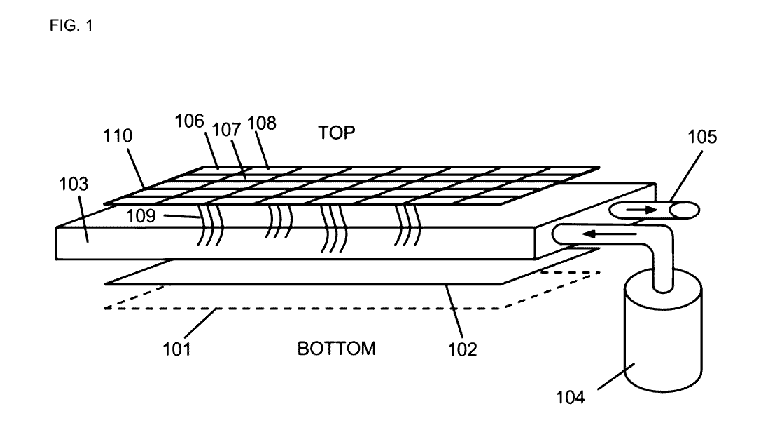

In some embodiments, DNA is synthesized first as a single strand (see FIG. 4), which may be converted to a double stranded molecule enzymatically post-synthesis using, for example, DNA polymerase. In some embodiments, to stabilize single-stranded DNA and prevent hairpins and other secondary structures from interfering with synthesis, single-stranded DNA-binding proteins (SSBs) or other stabilizers may be included in reagents (see FIG. 1). In some embodiments, strands complementary to object-attached DNA may be periodically synthesized using primers and enzymes such as, for example, DNA polymerase. In some embodiments, chemical-, or heat-, or photo-cleavable nucleotides may be incorporated at the 3′ end of surface-attached oligonucleotides to allow synthesis of the strand complementary to the bead-attached strand during each extension step. In some embodiments, the sequence extended beyond the chemical-, or heat-, or photo-cleavable nucleotides is cleaved from the surface-attached strand. In some embodiments, strands complementary to the solid object-attached sequence may be ligated using an enzymatic ligase or non-enzymatic chemical reaction.

In some embodiments, after synthesis of a desired predefined sequence an adapter sequence may be synthesized. In some embodiments, post-synthesis amplification of synthesized sequences may use a primer that is base-pair complementary to the adapter in conjunction with a primer equivalent to the solid object-attached primer used for synthesis. In some embodiments, primers may be modified by, for example, methylation to distinguish primer sequences from any other synthesized sequences. Ends of DNA molecules resulting from modified primers may be altered without altering any other sequence using enzymes including, but not limited to, FspEI, LpnPI, or MspJI (these examples being specific to methylated sequences).

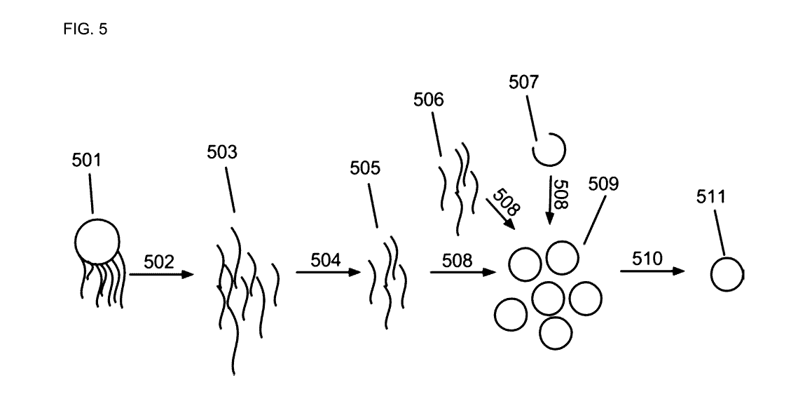

In some embodiments, DNA synthesized using the device and method herein may be assembled post-synthesis into larger sequences using such methods as are commonly known to those skilled in the art, including, but not limited to, restriction cloning and Gibson assembly (see FIG. 5). DNA synthesized using the devices and methods herein may be cloned into circular plasmid vectors or other vectors for transformation into host cell organisms and subsequent amplification and purification using such methods as are commonly known to those skilled in the art (see FIG. 5). In some embodiments, sequences synthesized by the device and method herein may be purified to remove sequence errors accrued during synthesis by methods including, but not limited to, filtration by sequence length and sequencing of single-sequence colonies or polonies using such methods as are commonly known to those skilled in the art (see FIG. 5).

In some embodiments, errors may accrue during synthesis due to errors in the template oligonucleotides on an oligonucleotide array. Because most sub-sequences synthesized during a particular cycle are used for hybridization during a subsequent cycle (see FIG. 4), errors in template oligonucleotides tend to cause failed hybridization during subsequent cycles. Failed hybridization leads to changes in total synthesized sequence length, and in some embodiments, length-altered molecules may be easily filtered out post-synthesis by methods commonly known to those skilled in the art. Failed hybridization does not lead to changes in total synthesized sequence length if a template oligonucleotide at one feature has an error which matches an error at another feature used in a subsequent cycle, and the chance of a template oligonucleotide occurring twice in the aforementioned manner is one-quarter the square of the chance of a single error in a template oligonucleotide. For example, if the error rate in template oligonucleotides is 1 per 1,000 nucleotides, the chance of non-length-altering synthesis errors is 1 per 4,000,000 nucleotides in some embodiments. The methods and devices provided herein thus provide a square error-rate improvement over the error rate of oligonucleotides used in many cases of the state of the art prior to post-synthesis purification.

### Certain Exemplary Embodiments

1. A method for synthesizing nucleic acids having a predefined or partially predefined sequence comprising:
a. controlled manipulation of solid objects with respect to a solid substrate comprising an oligonucleotide array;
b. base-pair hybridization of solid object-attached DNA strands to substrate-attached template strands comprising an oligonucleotide array feature;
c. enzymatic elongation of solid object-attached DNA strands complementary to the template strands comprising the oligonucleotide array;
d. repetition of aforementioned three steps for addition of subsequences of a predefined or partially sequence to solid object-attached DNA strands.

2. The method of embodiment 1 wherein the solid objects comprise a paramagnetic, superparamagnetic, ferromagnetic, dielectric, or other bead or disc about 100 microns, 50 microns, 10 microns, 5 microns, 1 micron or smaller in diameter.

3. The method of embodiment 1 or 2 wherein the location by sequence of template oligonucleotides attached to a solid substrate is stored on a computer.

4. The method of any of embodiments 1-3 wherein hybridization and elongation occurs in a liquid medium comprising proteins and enzymes such as polymerases, deoxynucleotide triphosphates, buffer, and other reagents.

5. The method of embodiment 4 wherein the liquid medium comprises single-stranded DNA stabilizers such as but not limited to single-stranded DNA binding proteins.

6. The method of any of embodiments 1-5 wherein template strands comprising an oligonucleotide array feature have a binding sequence complementary to solid object-attached DNA strands and a template sequence complementary to subsequent sequence to be synthesized.

7. The method of any of embodiments 1-6 wherein elongation is performed enzymatically by a DNA polymerase.

8. The method of any of embodiments 1-7 wherein the template strands comprising an oligonucleotide array feature have dideoxynucleotide, phosphate, inverted dT, or other 3′ chemical modifications to prevent extension by polymerases.

9. The method of any of embodiments 1-8 in which solid object-attached DNA is bound via biotin/streptavidin, maleic anhydride/amine, thiol/maleimide, or other covalent or noncovalent bond with or without a hydrocarbon, polyethylene glycol, or other spacer on either the DNA or solid object.

10. The method of any of embodiments 1-9 wherein the oligonucleotide attached to the solid object before any extensions comprises a universal or unique primer.

11. The method of any of embodiments 1-10 wherein the end of a predefined sequence comprises a universal adapter sequence.

12. The method of embodiment 10 wherein universal or unique primers comprise methylated or otherwise modified nucleotides which can be cleaved either bluntly or non-bluntly by methylation-(or other) specific restriction nucleases.

13. The method of any of embodiments 1-12 wherein the substrate-attached template strands comprise a chemical- or heat- or photo-cleavable nucleotide at the 3′ end.

14. The method of any of embodiments 1-13 wherein DNA strands complementary to solid-object attached sequences are ligated using an enzymatic ligase or non-enzymatic chemical reaction.

15. The method of any of embodiments 1-14 wherein dehybridization of oligonucleotides occurs by mechanical force rather thermal melting.

16. A nucleic acid assembly apparatus comprising a positioning system for solid objects, detection system, oligonucleotide template array, microfluidic network, and synthesis chamber.

17. A nucleic acid assembly apparatus of embodiment 16 wherein the positioning system comprises a computer or integrated circuit, which controls the positioning system in close-loop feedback control using feedback from the detection system.

18. A nucleic acid assembly apparatus of embodiment 16 or 17 wherein the number of independently controlled solid objects may be 1 or up to 10 or 100 or 1,000 or 10,000 or more.

19. A nucleic acid assembly apparatus of any of embodiments 16-18 wherein the positioning system positions solid objects by optoelectronic tweezers, optical tweezers with or without a micromirror array, dielectrophoresis, acoustic trapping, microfluidics, on- or off-chip magnetics, dielectrophoretic trapping with or without optical or microfluidic control, and mechanical motion of the stage or other methods.

20. A nucleic acid assembly apparatus of any of embodiments 16-19 wherein a detection system comprises a CMOS or CCD detector or other camera with or without additional optical components or other optical or non-optical detection devices.

21. A nucleic acid assembly apparatus of any of embodiments 16-20 wherein a microfluidics network comprises channels used to transfer solid objects to and from a synthesis chamber.

22. A nucleic acid assembly apparatus of any of embodiments 16-21 wherein an oligonucleotide template array comprises the full or partial set of unique sequences for a given oligonucleotide length.

23. A nucleic acid assembly apparatus of any of embodiments 16-22 wherein an oligonucleotide template array comprises longer or shorter sequences comprising tandem and homopolymer repeats.

24. A nucleic acid assembly apparatus of any of embodiments 16-23 wherein an oligonucleotide template array comprises locations at which a plurality of oligonucleotides comprises individual sequences or subsequences that have been randomized.

25. A nucleic acid assembly apparatus of any of embodiments 16-24 wherein an oligonucleotide template array comprises locations at which oligonucleotides partially or fully comprise non-canonical nucleotides other than adenine, guanine, cytosine, or thymine.

26. A nucleic acid assembly apparatus of any of embodiments 16-25 wherein an oligonucleotide template array comprises oligonucleotides with non-canonical universal nucleotides at the 3′ end.

27. A nucleic acid assembly apparatus of any of embodiments 16-27 wherein the oligonucleotide template array comprises oligonucleotides that vary from 4 to 200 or more nucleotides in length.

### Examples

### 1. Construction of a Magnetic Tweezers Apparatus

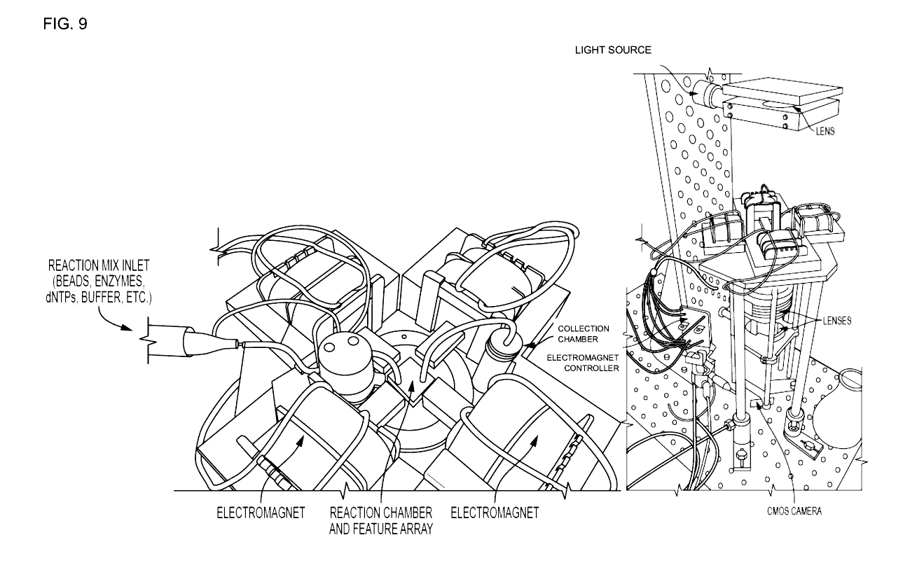

A magnetic tweezers apparatus (see FIG. 9) for controlled manipulation of superparamagnetic beads relative to an oligonucleotide template surface was constructed, as follows.

Electromagnets were constructed using repurposed transformers (from Radio Shack) in which “I” segments were removed and remaining “E” segments aligned to form a ferrous core. The electromagnets were adhered to a milled aluminum stage and mounted on a custom-built microscope. Control of the electromagnets was performed using a custom-built circuit via USB-computer interface. The microscope was constructed from components (from Thorlabs) for mounts and tube lenses, a Nikon Nikkor 35 mm f/1.4 manual lens as the objective lens, blue superbright LED as light source, and DMK24UJO03 monochrome camera (from Imaging Source) with USB output to a computer. The reaction chamber was produced by plasma-bonding molded PDMS on a glass slide. Tubing connects the chamber on one end to a syringe (controlled by a Harvard Apparatus Pump 11 Elite via computer-USB connection) loaded with Dynabeads (from Life Technologies), and to a collection vial on the other end.

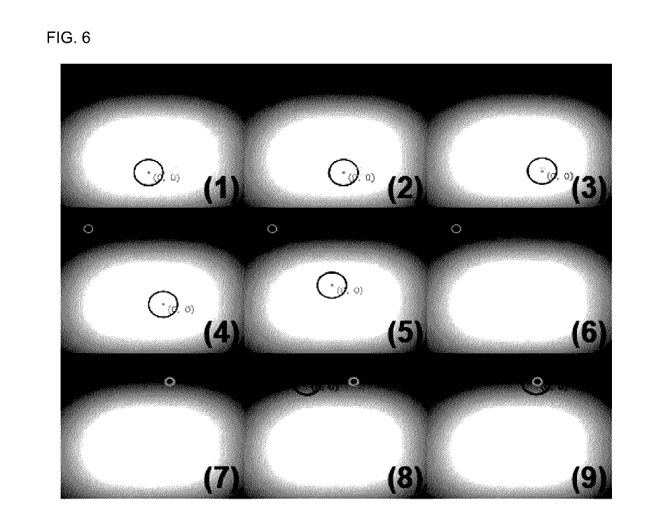

The position of a single 2.8 um Dynabead was controlled by computer software through PID control. The position of the bead (indicated by blue circle) was located by image processing, and the electromagnets were activated to pull the bead towards a target location (indicated by green circle). Images 1-9 in FIG. 6 show successive motion to 3 randomly generated targets before (images 1, 4, 7), during (images 2, 5, 8), and after (images 3, 6, 9) the bead's controlled motion.

### 2. Hybridization Between DNAs on the Surface of Two Solid Objects

Two biotinylated oligos (from IDT) with a designed 12 by hybridization region and 10 nt unhybridized region were attached to two samples of streptavidin-coated superparamagnetic beads (Dynabeads from Life Technologies). Bead-oligo 1, bead-oligo 2, and buffer were added in a tube. After brief mixing, aggregation of the beads due to base-pair hybridization were observed.

In a separate tube, bead-oligo 1 was mixed with liquid-phase oligo 2, and no aggregation of the beads were observed.

### 3. Synthesis of a Nucleic Acid

### Polymerase Extension for Ohms Attached to Solid Support

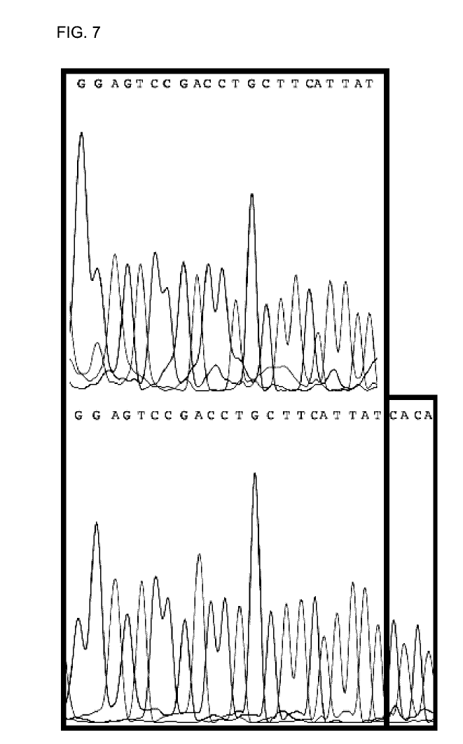

Oligos bound to Dynabeads were hybridized to a 12 nt, 3′ phosphate-capped template oligo with a designed 8 by hybridization region and combined with polymerase, dNTPs, and buffer. Product was run on an agarose gel and the band of expected length was excised and purified. The product was then blunt-ligated using a ssDNA ligase to add a longer adapter sequence (PCR priming site) to the 5′ end. A PCR reaction added a longer adapter sequence to the 3′ end and amplified the DNA. The DNA was again purified. The product was Sanger-sequenced, and the relevant section of the trace is shown (FIG. 7, bottom) alongside a no-template control (FIG. 7, top).

### Multiple Extension Cycles

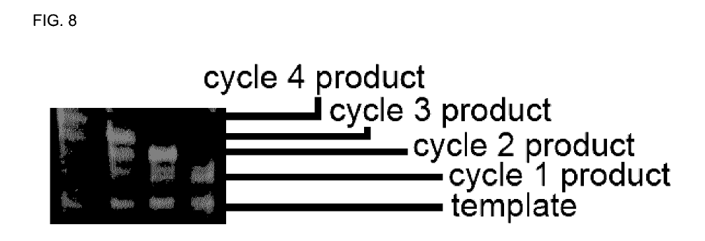

Polymerase extension reaction was run as described above with 3′-phosphate capped template oligos comprising a 12 by hybridization region and a 4 nt extension region. The beads were then isolated and washed in hot buffer to remove template molecules. The beads were then reacted with a second template oligo, washed, reacted with a third template oligo, washed, reacted with a fourth template oligo, and again washed. Samples after each cycle (before wash) were run on a denaturing PAGE gel (see FIG. 8). Each successive cycle shows an addition of approximately 4 nt to the product with lower than 100% yield giving rise to the lower bands in each lane).

While some embodiments are illustrated in the examples, it is apparent that they may be altered to provide other embodiments of the instant disclosure. Therefore, it will be appreciated that the scope of the invention is not be limited by the specific embodiments that have been represented by way of example.

1-15. (canceled)

16. A nucleic acid assembly apparatus comprising a positioning system for solid objects, detection system, oligonucleotide template array, microfluidic network, and synthesis chamber.

17. A nucleic acid assembly apparatus of claim 16 wherein a computer or integrated circuit controls a positioning system in close-loop feedback control using feedback from a detection system.

18. (canceled)

19. A nucleic acid assembly apparatus of claim 16 wherein the forces used to position solid objects are produced by optoelectronic tweezers, optical tweezers with or without a micromirror array, dielectrophoresis, acoustic trapping, microfluidics, on- or off-chip magnetics, dielectrophoretic trapping with or without optical or microfluidic control, and mechanical motion of the stage or other methods.

20. A nucleic acid assembly apparatus of claim 16 wherein a detection system comprises a CMOS or CCD detector or camera with or without additional optical components or other optical or non-optical detection devices.

21. A nucleic acid assembly apparatus of claim 16 wherein a microfluidics network comprises channels used to transfer solid objects to and from a synthesis chamber.

22-30. (canceled)

31. A method for synthesizing a nucleic acid having a predefined or partially predefined sequence, the method comprising:
(a) providing an oligonucleotide array, wherein the oligonucleotide array comprises multiple feature locations, and each feature location comprises a plurality of solid substrate bound oligonucleotides, wherein one end of the oligonucleotide attaches to the solid substrate, and the other end optionally comprises one or more modifications;
(b) providing a plurality of solid object attached oligonucleotides, wherein one end of the oligonucleotide attaches to a solid object, and the other end is compatible with enzymatic elongation;
(c) positioning the solid object with accompanying attached plurality of nucleic acids to a desired feature location of the oligonucleotide array;
(d) hybridizing the solid object attached oligonucleotides to the solid substrate bound oligonucleotides at the desired feature location to form a duplex region;
(e) extending the solid object attached oligonucleotides by enzymatic elongation of the duplex region in a reaction mixture comprising one or more enzymes, one or more nucleotide triphosphates, one or more buffers, and optionally one or more single-stranded DNA stabilizers;
(f) dehybridizing the solid object attached oligonucleotides from the solid substrate bound oligonucleotides at the desired feature location; and
(g) sequentially repeating steps (c) through (f).

32. The method of claim 31, wherein in (a) the oligonucleotide array comprises around 10, 100, 1000, 10000, 100000, 1000000 or more feature locations.

33. (canceled)

34. The method of claim 31, wherein in (a) each solid substrate bound oligonucleotide is bound to the solid substrate surface at its 5′ end.

35. The method of claim 31, wherein in (a) the one or more modification is at the 3′ end of each solid substrate bound oligonucleotide.

36. The method of claim 31, wherein in (a) the one or more modification comprises a cap, optionally a dideoxynucleotide, phosphate, inverted dT, or other 3′ chemical modifications to prevent the solid substrate bound oligonucleotides from enzymatic elongation.

37. The method of claim 31, wherein in (a) the one or more modification comprises a chemical-, or heat-, or photo-cleavable nucleotide, which allows enzyme elongation of the solid substrate bound oligonucleotides.

38. The method of claim 31, wherein in (a) the solid substrate is a glass or silicon substrate.

39. The method of claim 31, wherein in (b) the solid object is a paramagnetic, superparamagnetic, ferromagnetic, dielectric, or other bead or disc, optionally about 100 microns, 50 microns, 10 microns, 5 microns, or 1 micron or smaller in diameter.

40. The method of claim 31 wherein the number of independently controlled solid objects may be 1 or up to 10 or 100 or 1,000 or 10,000 or more.

41. The method of claim 31, wherein in (b) solid object attached oligonucleotides are bound to solid objects via biotin/streptavidin, maleic anhydride/amine, thiol/maleimide, or other covalent or noncovalent bond with or without a hydrocarbon, polyethylene glycol, or other spacer on either the oligonucleotides or solid objects.

42. The method of claim 31, wherein in (b) each solid object attached oligonucleotide attaches to the solid object at its 5′ end.

43. The method of claim 31, wherein in (c) the solid object attached oligonucleotides are moved to position the plurality of the solid object attached oligonucleotides to a desired feature location of the oligonucleotide array.

44. The method of claim 31, wherein in (d) the duplex region comprises 2 or more canonical or non-canonical base pairs.

45. (canceled)

46. The method of claim 31, wherein in (e) the single-stranded DNA stabilizer is a single-stranded DNA binding protein.

47. The method of claim 31, wherein in (e) the enzyme is DNA polymerase.

48. The method of claim 31, wherein in (e) the nucleotide triphosphate is a non-canonical nucleotide triphosphate.

49. The method of claim 31, wherein each solid object attached oligonucleotide before any extensions comprises a universal primer, or a sequence region that is complementary to a universal primer, and/or wherein the predefined sequence comprises at its 3′ end a universal adapter sequence or a sequence region that is complementary to a universal primer.

50. (canceled)

51. The method of claim 49, wherein the universal primer comprises methylated or otherwise modified nucleotides, which can be cleaved either bluntly or non-bluntly by methylation (or other) specific restriction nucleases.

52. The method of claim 31, wherein in (f) dehybridization of the solid object attached oligonucleotides from the solid substrate bound oligonucleotides is by mechanical force.

53. The method of claim 31, wherein DNA strands complementary to solid-object attached sequences are ligated using an enzymatic ligase or non-enzymatic chemical reaction.

54. The method of claim 31, wherein an oligonucleotide array comprises the full or partial set of unique sequences for a given oligonucleotide length.

55. The method of claim 31, wherein the oligonucleotide array comprises longer or shorter sequences comprising tandem repeats and/or homopolymer repeats.

56. The method of claim 31, wherein the oligonucleotide array comprises feature locations at which a plurality of oligonucleotides comprise individual sequences or subsequences that have been randomized.

57. The method of claim 31, wherein the oligonucleotide array comprises feature locations at which oligonucleotides partially or fully comprise non-canonical nucleotides other than adenine, guanine, cytosine, or thymine.

58. (canceled)

59. The method of claim 31, wherein the oligonucleotide array comprises oligonucleotides that vary in length from 4 to 200 or more nucleotides in length.
